(19) United States
(12) Patent Application Publication
(10) Pub. No.: US 2007/0122429 A1
(43) Pub. Date: May 31, 2007

(54) Designed antigens to elicit neutralizing antibodies against sterically restricted antigen and method of using same

(76) Inventors: Michael S. Kay, Salt Lake City, UT (US); Brett Welch, Draper, UT (US)

Correspondence Address: TRASK BRITT, P.O. BOX 2550, SALT LAKE CITY, UT, 84110, US

(21) Appl. No.: 11/484,101

(22) Filed: Jul. 11, 2006

Related U.S. Application Data
(60) Provisional application No. 60/698,573, filed on Jul. 11, 2005.

(51) Int. Cl. A61K 39/395; A61K 39/12

(52) U.S. Cl. 424/204.1; 424/130.1

(57) ABSTRACT

The invention relates to the production of sterically restricted antigens, antibodies useful for the recognition of sterically restricted antigens, and methods of identifying and/or using the same. The invention further relates to methods of using the sterically restricted antigens and antibodies to treat a disease or to prevent infection with a disease.

### CROSS-REFERENCE TO RELATED APPLICATION

Pursuant to the provisions of 35 U.S.C. §119(e), this application claims the benefit of the filing date of Provisional Patent Application Ser. No. 60/525,562, filed Jul. 11, 2006, the contents of the entirety of which are hereby incorporated by this reference.

### STATEMENT REGARDING FEDERALLY SPONSORED RESEARCH OR DEVELOPMENT

Work described herein was supported, in part, by National Institutes of Health Grant GM066521. The United States government may have certain rights in the invention.

### TECHNICAL FIELD

This invention relates to biotechnology, more specifically to the production of sterically restricted antigens, antibodies useful for the recognition of sterically restricted antigens, and methods of identifying and/or using the same. The invention further relates to methods of using the sterically restricted antigens and antibodies to treat a disease or to prevent infection with a disease.

### BACKGROUND

Since the discovery of human immunodeficiency virus type 1 (HIV-1) two decades ago, over 20 million deaths have been attributed to Acquired Immune Deficiency Syndrome (AIDS). Currently, over 36 million people worldwide are infected with the virus, corresponding to one HIV-positive person for every 200 people in the world. At the end of 2000, there were 16 anti-HIV-1 drugs approved by the Food and Drug Administration targeting only two viral proteins. The two current viral targets are reverse transcriptase, which is responsible for transcribing the HIV-1 RNA genome to DNA, and the protease, which processes the HIV-1 Gag/Pol polyprotein and the subsequent Gag protein. Because of the high rate of viral turnover and the error-prone nature of reverse transcriptase, viruses resistant to these small-molecule drugs often emerge.

Currently in the United States, combination therapy, for example, in which three or more drugs are administered concomitantly, is a routine treatment. Although combination therapy is often successful at lowering viral load, there are significant problems associated with it. Some patients develop immediate adverse effects and are therefore intolerant to the available drugs. Even those patients who are more tolerant face expensive, arduous treatment. Further, some patients harbor viral strains resistant to several drugs, and long-term adverse effects of treatment can develop. Also, because of increasing viral resistance, the threat of an outbreak of a virus immune to all available drugs is rising. Therefore, drugs that target an additional step of the viral life cycle, such as viral entry, would be useful, especially if they have fewer adverse side effects and are less susceptible to viral resistance than current therapies. HIV-1 envelope glycoprotein (Env), which promotes viral membrane fusion through receptor-mediated conformational changes and determines viral tropism, is an attractive target because it is expressed on the surface of both virus and infected cells. In addition, the envelope glycoprotein is required for viral entry into the cell.

Viruses frequently synthesize their fusion glycoproteins in an inactive form. In this state, the fusion glycoprotein adopts a thermodynamically stable conformation. Subsequently, the fusion glycoprotein is proteolytically processed into two subunits, a surface subunit and a transmembrane subunit. The protein then waits in a metastable state for the appropriate activation signal. After the signal arrives, the glycoprotein unleashes its fusion potential (for review, see, Earp L. J, Delos S. E, Park H. E., White J. M., (2005) The Many Mechanisms of Viral Membrane Fusion Proteins, Curr Top Microbiol Immunol. 285:25-66). No additional energy, such as ATP hydrolysis, is required. Prior to fusion, the HIV envelope protein (gp41) spans both membranes, and is in a conformation referred to as a “pre-hairpin intermediate,” that is vulnerable to inhibition for many minutes (approximately 15 minutes). The protein then adopts its most stable fold, referred to as a “trimer-of-hairpins,” and utilizes the energy harnessed through acquisition of this state or conformation to promote fusion of the two membranes.

The HIV Env protein is initially synthesized as a single polypeptide precursor, gp160, and is subsequently proteolytically cleaved into a heavily glycosylated surface subunit known as gp120 and a transmembrane subunit known as gp41. The two subunits are then associated by non-covalent bonds in an oligomeric structure on the surface of the virion (Decroly, et al. 1994. The convertases furin and PC1 can both cleave the human immunodeficiency virus (HIV)-1 envelope glycoprotein gp160 into gp120 (HIV-1 SU) and gp41 (HIV-I TM) (J. Biol. Chem. 269:12240-12247; Hallenberger, et al. 1992. Inhibition of furin-mediated cleavage activation of HIV-1 glycoprotein gp160. Nature 360:358-361; Morikawa, et al. 1993. Legitimate and illegitimate cleavage of human immunodeficiency virus glycoproteins by furin. J. Virol. 67:3601-3604). On the target cell surface, the gp120 surface protein binds to CD4 and a co-receptor, leading to a conformational change in gp120 that alters gp120-gp41 interactions (Kwong, et al. 1998. Structure of an HIV gp120 envelope glycoprotein in complex with the CD4 receptor and a neutralizing human antibody; Nature 393:648-659; Wu, et al. 1996). CD4-induced interaction of primary HIV-1 gp120 glycoproteins with the chemokine receptor CCR-5. Nature 384:179-183). This binding event triggers membrane fusion, which requires functions of the gp41 ectodomain. The gp41 ectodomain comprises an amino-terminal and carboxy-terminal leucine/isoleucine heptad repeat domain with a helical structure (N-peptide helix, N51, and C-peptide helix, C43, respectively).

The trimer-of-hairpins is a common structural element involved in the fusion process of many enveloped viruses, suggesting a critical role for this motif in promoting membrane fusion (D. C. Chan and P. S. Kim, Cell 93, 681 (1998); M. P. D'Souza, J. S. Cairns and S. F. Plaeger, JAMA 284, 215 (2000); F. Hughson, Curr. Biol. 7, R565 (1997)). In HIV-1 gp41, the core of the trimer-of-hairpins is a bundle of six α-helices (FIG. 1B): three α-helices (formed by the COOH-terminal regions of three gp41 ectodomains) pack in an antiparallel manner against a central, three-stranded coiled coil (formed by the NH₂-terminal regions of the gp41 molecules) (Zhao, et al. Proc. Natl. Acad. Sci. U.S.A. 97, 14172 (2000); Singh, et al. J. Mol. Biol. 290, 1031 (1999); Lu, et al. Nature Struct. Biol. 2, 1075 (1995)). The fusion peptide region, which inserts into the cellular membrane, is located at the extreme NH₂-terminus of gp41, and the COOH-terminal region is adjacent to the transmembrane helix anchored in the viral membrane. Thus, the trimer-of-hairpins motif brings the two membranes together. The trimer-of-hairpins is a coiled-coil structure composed of internal triple-stranded N-peptide helices paired with antiparallel outer C-peptide helices packed along hydrophobic grooves, thus forming a six-helix bundle (Munoz-Barroso, et al. (1998) J. Cell Biol. 140, 315-323, McCaffrey, et al. (2004) J. Virol. 78, 3279-3295). The adoption of this trimer-of-hairpins conformation is believed to provide the energy necessary to drive fusion of the viral particle with the host cell.

The Env glycoprotein, (gp120 and gp41), is an attractive target for the development of antiviral agents for at least two reasons: (i) it is present on the surface of both virus and infected cells, and (ii) it mediates the initial stages of viral infection, attachment and membrane fusion, rather than the later, post-entry stages of reverse transcription and proteolysis (Wyatt, R. & Sodroski, J. (1998) Science 280, 1884-1888; Freed, E. O. & Martin, M. A. (2001) in Field's Virology, ed. Howley, P. M. (Lippincott, Philadelphia), pp. 1971-2041; Biscone, M., Pierson, T. & Doms, R. (2002) Curr. Opin. Pharmacol. 2, 529).

In theory, epitopes derived from either subunit are prime targets for the development of antiviral therapeutics that can either block HIV-1 entry or destroy infected cells. Attempts to develop such compounds have been largely unsuccessful for two reasons. First, the surface of Env is poorly structured and highly variable owing to the high degree of glycosylation and the presence of many flexible, non-conserved peptide loops. Second, the high HIV-1 mutation rate permits Env to escape inhibition by strain-specific antiviral agents and antibodies.

However, the ectodomains of gp41 provide well conserved epitopes that are crucial to Env function. During the fusion process, the gp41 N-terminal regions become transiently accessible to inhibitory compounds (Furuta, et al. (1998) Nat. Struct. Biol. 5, 276-279, Melikyan, et al. (2000) J. Cell Biol. 151, 413-423). In this transient state, the pre-hairpin intermediate, the gp41 N terminus is inserted in the target cell membrane and the N-terminal coiled coil is exposed, but the trimer-of-hairpins has not yet formed (Eckert, D. M. & Kim, P. S. (2001) Annu. Rev. Biochem. 70, 777-810).

Because of its central role in mediating viral entry, the N-trimer region of gp41 is a key vaccine target. Extensive efforts to discover potent and broadly neutralizing antibodies (Abs) against the N-trimer region have, thus far, been unsuccessful. In contrast, previous results suggest that the C-peptide region is accessible during fusion, demonstrating that the N— and C-peptide regions are in structurally distinct environments.

Recently, Merck has reported preliminary results on an antibody that binds to the N-trimer region and possesses neutralizing activity against some HIV strains (52). No detailed information on this Ab has yet been published, but it will be interesting to see whether or how this Ab circumvents steric restriction (e.g., high affinity Ab that can tolerate several hundred-fold loss in activity, extended variable loops, specific targeting of a subsite in the N-trimer).

Peptides derived from the gp41 C-terminal region (C-peptides) can bind to the exposed coiled coil and block the proper formation of the trimer-of-hairpins, thus preventing membrane fusion (Chan, et al. (1998) Proc. Natl. Acad. Sci. USA 95, 15613-15617; Wild, et al. (1994) Proc. Natl. Acad. Sci. USA 91, 9770-9774;, Jiang, et al. (1993) Nature 365, 113). C-peptides can be potent inhibitors of HIV-1 entry, with IC₅₀ values as low as 1 nM in vitro. Two C-peptides, T20 and T1249, are currently in clinical trials and show antiviral activity in humans (Moore, J. P. & Stevenson, M. (2000) Nat. Rev. Mol. Cell Biol. 1, 40-49; Biscone, M., Pierson, T. & Doms, R. (2002) Curr. Opin. Pharmacol. 2, 529; Kilby, et al. (1998) Nat. Med. 4, 1302-1307). However, raising an effective neutralizing antibody response has been much more elusive. A safe HIV-1 vaccine targeting N-trimer formation could provide an effective treatment and may provide a vaccine that would prevent or decrease the rate of new infections in the world. Because many enveloped viruses likely use the same mechanism of entry, similar therapeutic strategies may be effective against a wide range of viral diseases.

### SUMMARY OF THE INVENTION

The invention relates to the production of sterically restricted antigens, antibodies useful for the recognition of sterically restricted antigens, and methods of identifying and/or using the same.

In one exemplary embodiment, the present invention provides an antigen linked to a sterically restrictive agent, which may be used to induce an immune response in a subject, to generate antibodies capable of recognizing both the sterically restricted antigen and the antigen in its wild-type state, and to prevent the function of the molecule from which the antigen is derived, and/or as a research tool for the study of viral fusion.

In one exemplary embodiment, the present invention provides an HIV N-trimer (e.g. N-Protein, 5-helix, IZN36, N_{CCG}-gp41) linked to a sterically restrictive agent (“cargo”), which may be used to induce an immune response in a subject, to generate, for example, monoclonal antibodies capable of recognizing the sterically restricted antigen, or as an research tool for the study of HIV infection. The monoclonal antibodies may bind to the sterically restricted antigen and/or prevent the function of the molecule from which the antigen is derived.

Another exemplary embodiment of the present invention provides an antibody that specifically binds a sterically restricted antigen and a method of identifying the same. One exemplary embodiment comprises an antibody that recognizes the N-trimer region of a gp41 protein that is capable of overcoming the apparent steric occlusion of this region. The anitbody may bind to the sterically restricted antigen and/or prevent the function of the molecule from which the antigen is derived.

In yet another exemplary embodiment, the present invention provides a monoclonal antibody, preferably a humanized monoclonal antibody, that specifically binds the antigen (e.g., N-trimer region of a gp41 protein) that is capable of overcoming an apparent steric restriction. The monoclonal antibody may bind to the sterically restricted antigen and/or prevent the function of the molecule from which the antigen is derived.

In yet another exemplary embodiment, the present invention provides a pharmaceutical preparation comprising an antigen linked to a sterically restrictive agent and a pharmaceutically acceptable excipient, diluent and/or carrier. In yet another exemplary embodiment, the present invention provides an antibody having the ability to access a sterically restricted antigen, wherein the Ab specifically binds the antigen and is capable of overcoming an apparent steric restriction. The antibody may bind to the sterically restricted antigen and/or prevent the function of the molecule from which the antigen is derived.

In another exemplary embodiment, the present invention provides methods for generating an immune response in a subject comprising administering to a subject the pharmaceutical preparations outlined supra. In a further exemplary embodiment, the antigen portion of the sterically restricted antigen is associated with a disease. The immune response of the subject to the sterically restricted antigen generates an antibody to the antigen protion and the binding of the antibody to the wild-type antigen in vivo treats the disease or prevents infection with the disease. Thus, another exemplary embodiment of the invention provides methods for treating a disease or preventing infection with a disease through the treatment of a subject having the disease or believed to be a risk for infection with the disease with the sterically restricted antigens of the present invention. By way of a non-limiting example, the disease may be AIDS.

In a further exemplary embodiment of the present invention, the treatment of the subject with the sterically restricted antigen immunizes the subject against (or induces an immune response against) the disease associated with the antigen portion of the sterically restricted antigen.

In yet another exemplary embodiment, the present invention provides an antibody having at least one long (relative to the length in an average antibody from the same species) Complementarity Determining Region (CDR) that specifically binds the antigen, for example, the N-trimer region of a gp41 protein, and that is able to overcome an apparent steric restriction. The antibidody may bind to the sterically restricted antigen and/or prevent the function of the molecule from which the antigen is derived.

In yet another exemplary embodiment, the present invention provides an antibody having structural characteristics that specifically binds an antigen, for example, the N-trimer region of a gp4l protein, wherein the structural characteristics are capable of overcoming a steric restriction of the antigen. The binding of the antibody to the sterically restricted antigen may prevent the function of the molecule from which the antigen is derived.

In an exemplary embodiment, the sterically restrictive agent ranges in size from about 20 Å to 10 um (e.g., ranging in size from small proteins to large gold or agarose particles). Examples include, but are not limited to: proteins (preferably natural proteins that are not immunogenic or have low immunogenic potential), such as Maltose Binding Protein, Green Fluorescent Protein, Human Serum Albumin, Bovine serum albumin, Mouse serum albumin, Rabbit serum albumin, Ovalbumin, keyhole limpet hemacyanin, biocompatible polymers (see, U.S. Pat. No. 6,913,936), biocompatible nanoparticles, Streptavidin, and Immunoglobulin domains (e.g., ranging from ˜20 to 100 Å in diameter), and proteins having a modification such as, but not limited to, polyethylene glycol (PEG) modifications (e.g., ranging from 1 kD to 100 kD), gold particles (e.g., from about 1 nm to about 30 nm), magnetic beads (e.g., about 1 um), and combinations thereof. A sterically restrictive agent may be attached or linked to the antigen (epitope) at one or more locations, for example, the IZN36 antigen may have a gold particle attached at one end and a protein on the other.

In another exemplary embodiment, the antigen is embedded in liposomes, which may be approximately 100 nm in diameter, to produce a sterically restricted antigen. This embodiment may be used to mimic the natural context of a membrane associated antigen.

Another aspect of the present invention relates to a substantially pure antibody (Ab), such as a monoclonal or polyclonal antibody, or antibody derivative, that specifically recognizes and binds to a sterically restricted antigen, for example, the N-trimer region of gp41, in vivo. The binding of the antibody to the sterically restricted antigen may prevent the function of the molecule from which the antigen is derived.

A further aspect of the present invention relates to a method of treating or preventing a disease in a subject, the method comprising providing to the subject a pharamaceutical composition comprising an antibody according to the present invention; and monitoring the presence of the disease state in the subject. By way of non-limiting example, the disease may be AIDS.

In another exemplary embodiment, the present invention provides a method of screening existing Ab libraries (e.g., Fab and/or scFv libraries created via phage display) for Abs likely able to overcome the steric restriction, for example, the Ab library may be enriched for Abs having longer CDR region.

In another exemplary embodiment, the present invention provides a recombinant antigen (e.g., IZN36) that is expressed with a sterically restrictive agent comprising a protein (providing steric restriction) linked/fused to the antigen. For example, a sterically restrictive agent linked to the C-terminus of IZN36. Optionally, a targeting moiety may be attached, such as by biotinylating a cysteine residue, allowing the recombinant protein to be bound to the surface of a SA Biacore chip. In one exemplary embodiment, IZN36 is linked to MBP as the sterically restrictive agent. C37 is a His-tagged version of the previously characterized synthetic peptide C34, which, in itself, is a section N-trimer region of HIV-1 gp41 (Hamburger et al (2005) J. of Bio. Chem. 280(13); 12567-12572). This embodiment may be used to measure the ability of proteins or compounds (e.g., a C-peptide inhibitor, Ab libraries, Ab, and derivatives thereof (e.g., Fab and/or scFv libraries)) to overcome the steric restriction. The method is believed to produce results similar to those observed with IC₅₀ in the cell-cell and viral assays using the cargo-C37 fusion proteins. Optionally, this method may also be used to further delineate the nature and size of the steric restriction observed in the N-trimer (e.g., reduce the affinity of MBP-C37 while retaining the affinity of C37). The method is preferably used to screen Ab libraries (e.g., Fab and/or scFv libraries created via phage display) for Abs able to overcome the steric restriction.

In another exemplary embodiment, the present invention provides an antigen, e.g., the N-trimer IZN36, with cargo attached to both ends of the antigen. In another embodiment, cargo may be a peptide, and, optionally, the peptide may be PEGylated to alter the immunogenic properties of the peptide.

In yet another exemplary embodiment, the present invention provides a method of generating an antibody, an immunological response, and/or a sterically restricted surface peptide antigen. For example, surface peptide antigens on virulent phase I Coxiella burnetii (T. Hackstadt, (1988) Steric Hindrance of Antibody Binding to Surface Proteins of Coxiella burnetii by Phase I Lipopolysaccharide, Infection and Immunity, 56(4):802-807), viral fusion cores, such as coronaviruses, influenza virus, severe acute respiratory syndrome virus (see, Xu et al. (2004) Structural Basis for Coronavirus-mediated Membrane Fusion: Crystal Structure of Mouse Hepatitis Virus Spike Protein Fusion Core, J. Biol. Chem. 279(29):30514-30522).

The invention also provides pharmaceutical compositions and methods of manufacturing the pharmaceutical composition, which can be administered to a patient to achieve a therapeutic effect.

### BRIEF DESCRIPTION OF DRAWINGS

FIGS. 1A to 1C illustrate the targeting of HIV-1 membrane fusion. FIG. 1A illustrates a schematic of HIV-1 membrane fusion depicting events that promote formation of the gp41 trimer-of-hairpins (see, reference 1). The NH₂-terminal fusion peptide of gp41, inaccessible in the native state, inserts into target cell membranes following gp120 interaction with CD4 and co-receptors. Formation of the prehairpin intermediate exposes the NH₂-terminal coiled coil, the target of C-peptide inhibition. This transient structure collapses into the trimer-of-hairpins state that brings the membranes into close apposition for fusion. FIG. 1B illustrates lateral (left) and axial (right) views of a ribbon diagram representing the core of the gp41 trimer-of-hairpins. The ribbon diagram is derived from the crystal structure of a six-helix bundle formed by N36 (N-peptide) and C34 (C-peptide) (7). FIG. 1C illustrates a schematic model of the designed protein 5-Helix. Three N-peptide segments (N40) and two C-peptide segments (C38) are alternately linked (N—C—N—C—N) using short Gly/Ser peptide sequences (21). The sequences of each segment in single-letter amino acid code are: N40, QLLSGIVQQQNNLLRAIEAQQHLLQLTVWGIKQLQARILA (SEQ ID NO:1); C38, HTTWMEWDREINNYTSLIHSLIEESQNQQEKNEQELLE (SEQ ID NO:2); N-to-C linker, GGSGG (SEQ ID NO:3); and C-to-N linker, GSSGG (SEQ ID NO:4).

FIGS. 2A and 2B illustrate the inhibitory activity of C37 and C37 fusion proteins. Data points are averages of at least quadruplicate measurements. Data are normalized to uninhibited fusion activity. a, cell-cell fusion assay (HXB2 Env). S.E. of each point is <0.05. b, viral infectivity assay (HXB2 Env). S.E. of each point is <0.1. An overshoot is observed at low inhibitor concentrations (data above 1.0). Fitting the viral infectivity data to a simple Langmuir equation with a fixed zero inhibitor point produces noticeable deviation from the data near the zero point because of this overshoot. Fitting the data without fixing the zero inhibitor point (as done in this study) improves the quality of the fit but does not significantly affect the relative IC₅₀ values of the inhibitors. MBP-C37 is represented by an “x”; Mb-C37 is represented by closed squares; GFP-C37 is represented by open squares; Ub-C37 is represented by triangles; BPTI-C37 is represented by inverted triangles; and C37 is represented by open circles.

FIG. 3 illustrates the binding of C37 and C37 fusion inhibitors to IZN36 measured by SPR. Responses for representative inhibitors were normalized and overlaid to facilitate their comparison (thick traces). Fits to the interaction model are included (thin traces). Inset, interaction of control proteins (no C37) with IZN36 surface.

### DETAILED DESCRIPTION OF THE INVENTION

Despite the currently available drugs, there are increasing problems with long-term toxicity, high cost, difficulties adhering to treatment regimens, and emergence of multiple, drug-resistant viral strains. Accordingly, therapeutics are needed that target conserved regions of proteins involved in different viral life cycle events; and, thus, are likely to be active against isolates resistant to current drugs. More preferable, are therapeutics or vaccines that can eliminate infected cells, thereby reducing persistent and latent reservoirs of the virus. Even more preferably, are therapeutics that may treat or prevent viral infection.

As used herein and in the appended claims, the singular forms “a”, “an”, and “the” include plural reference unless the context clearly dictates otherwise. For example, reference to “a steric agent” includes a plurality of such steric agents, and reference to the “antibody” is a reference to one or more antibodies and equivalents thereof known to those skilled in the art, and so forth.

As used herein “Antigen” means an immunogenic region of a peptide having at least one epitope.

As used herein “Peptide,” “Polypeptide” and “Protein” include polymers of two or more amino acids, and includes post-translational-modification. No distinction, based on length, is intended between a peptide, a polypeptide or a protein.

As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.”

As used herein, “about” means reasonably close to, a little more or less than the stated number or amount, or approximately.

Ranges can be expressed herein as from “about” one particular value, and/or to “about” another particular value. When such a range is expressed, another embodiment includes each number from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, “Liposome” means a synthetic membrane vesicle made from phospholipids. Liposomes are commonly used for in vitro study of membrane-defined events, such as, transport or delivery of substances to a cell.

As used herein, “Monoclonal Antibody” means an antibody produced from a cultured cell, typically a hybridoma, wherein the antibody consist of a single molecular entity from a single clone of antibody-producing cells. Hybridomas usually result from the fusion of mouse spleen cells responding to a particular antigen with an immortalized mouse myeloma cell line. Monoclonal antibodies may also be generated from a phage display library and expressed in animal cell culture or E. coli (scFv antibody fragments).

As used herein, “Polyclonal Antibody” means an antibody preparation obtained from animal whole serum which has antibodies that recognize many different epitopes.

As used herein, “Treat,” “Treating,” or “Treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

As will be understood using the guidance of the specification, a sterically restricted antigen designed to elicit an immunological response (e.g., a vaccine) in a subject, such as a human, mouse, rat, goat, sheep, rabbit or other animal, may be combined with an adjuvant.

Pharmaceutical compositions of the invention may comprise, for example, a sterically restricted antigen (e.g., N-trimer), or mimetics thereof, ribozymes capable of recognizing and cleaving the sterically restricted antigen, and/or antibodies capable of recognizing the sterically restricted antigen. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

In addition to the active ingredients, the pharmaceutical compositions may contain suitable pharmaceutically-acceptable carriers comprising excipients and auxiliaries which facilitate processing of the active compounds into preparations which can be used pharmaceutically. Pharmaceutical compositions of the invention may be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for use may be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or peptide fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and peptides such as gelatin and collagen. If desired, disintegrating or solubilizing agents can be added, such as the cross-linked polyvinyl pyrrolidone, agar, alginic acid, or a salt thereof, such as sodium alginate. Dyestuffs or pigments may be added where desirable.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition may be provided as a salt, which tend to be more soluble in aqueous or other protonic solvents, and may be formed with many acids, including but not limited to, hydrochloric, sulfuric, acetic, lactic, tartaric, malic, succinic, etc. In an exemplary embodiment, the invention provides a sterically restricted N-trimer in an aqueous solution buffered to a pH of between about 2 to about 11, more preferably, between about 3 and about 9, more preferably, between about 5 and about 8, even more preferably the solution is buffered to the appropriate physiological pH for the subject (e.g., about 7.4).

Further details regarding techniques for formulation and administration may be found in Remington's Pharmaceutical Sciences (Maack Publishing Co., Easton, Pa.). After pharmaceutical compositions have been prepared, they may be placed in an appropriate container and labeled for treatment of an indicated condition. Such labeling would include amount, frequency, and method of administration.

The antigens of the invention (e.g., an N-trimer mimic) may be modified by attaching substances, for example, cargo molecules, to a primary amine, which are found primarily on lysine residues. Lysine residues are easily modified due to their reactivity and their typical location on the surface of proteins. Another common target is sulfhydryls, which exist in proteins in reducing conditions. A sulfhydryl group may be introduced into a protein sequence by reduction of disulfides, chemical modification of primary amines or point mutation to introduce cysteine residues. Other common targets include, but are not limited to, carboxyls and carbohydrates. Carboxyls, like primary amines, are abundant and easily accessible. Coupling a cargo molecule to a carboxyl is frequently facilitated by the use of a cross-linker, which are known in the art (e.g., EDAC). Carbohydrate moieties, present on glycoproteins, may be modified by adding additional carbohydrate moieties or other substances, typically, by converting the carbohydrate into an aldehyde.

The antigens of the invention (e.g., an N-trimer mimic) may also be modified by the creation of fusion constructs (chimeric molecules) containing the antigen. Proteins commonly used in fusion constructs include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion constructions, including histidine (His) tags, FLAG tags, influenza hemagglutinin (HA) tags, Myc tags, VSV-G tags, and thioredoxin (Trx) tags. Other fusion constructions may include maltose binding protein (MBP), S-tag, Lex, a DNA binding domain (DBD) fusion (e.g., a GAL4 DNA binding domain fusion), and herpes simplex virus (HSV) BP16. A fusion construct may also be engineered to contain a cleavage site, for example, located in the linker sequence. Preferably, a protein fused to an N-trimer or C-peptide of the invention is not highly antigenic, more preferably, it has a low antigenic potential.

A fusion protein may be synthesized chemically, as is known in the art. Recombinant DNA methods can be used to prepare fusion proteins, for example, by making a DNA construct which comprises a coding sequence and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz BioTechnology (Santa Cruz, Calif.), MBL International Corporation (MIC; Watertown, Mass.), and Quantum Biotechnologies (Montreal, Canada; 1-888-DNA-KITS).

PEG groups may be used to directly or indirectly PEGylate either the antigen or the restrictive agent (cargo), for example, PEGylation of the ends of the N-trimer, thus providing steric bulk and increased half-life of the molecule. PEGylated peptide synthesis reagents can specifically control the placement of PEG groups within the IZ portion of IZN36, controlling the stringency of the steric restriction. These methods may also be applied to other antigens (e.g., N-trimer mimics, such as IZN17) in order to select for N-trimer pocket-specific Abs and/or to increase the steric restriction surrounding the antigen.

As will be recognized in light of the present disclosure, the invention overcomes limitations of current mimics of the N-trimer and other sterically restricted antigens, which do not specifically select for Abs having the desired characteristics. In contrast, using the sterically restricted N-trimer mimic of the present invention as an antigen, Abs able to overcome the steric restriction may be selected. For example, an artificially designed antigen (IZN36) that mimics the N-trimer may be provided with a steric restriction and used as an antigen to select for Abs that circumvent this protection. The designed, sterically restricted, N-trimer mimic of the invention provides the ability to select for such an antibody.

Using the N-trimer and/or C-peptide, linked to sterically restrictive agent (i.e., cargo), described herein, anti-gp41 antibodies may be produced by any standard technique. The designed, sterically restricted, N-trimer (or C-peptide) is preferably purified by standard techniques and is used to immunize rabbits. The antisera obtained is then itself purified, for example, on a GST-sterically restricted N-trimer affinity column and is shown to specifically identify the N-trimer region, for example, by Western blotting.

Polypeptides for antibody production may be produced by recombinant or peptide synthetic techniques (see, e.g., Solid Phase Peptide Synthesis, supra; Ausubel et al., supra).

For polyclonal antisera, the cargo may, if desired, be a carrier protein, such as KLH as described in Ausubel et al, supra. The KLH-N-trimer is mixed with Freund's adjuvant and injected into guinea pigs, rats, goats or preferably rabbits. Antibodies may be purified by any method of affinity chromatography.

Alternatively, monoclonal antibodies may be prepared using a sterically restricted N-trimer, or immunogenic fragment or analog thereof, and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In: Monoclonal Antibodies and T Cell Hybridomas, Elsevier, N.Y., 1981; Ausubel et al., supra).

In addition antibody fragments which contain specific binding sites for the N-trimer region may be generated. Preferably, the antibodies or antibody fragments bind with high specificity to the sterically restricted N-trimer sequence. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse, W. D. et al (1989) Science 256:1275-1281).

Once produced, the polyclonal or monoclonal antibody is tested for specific recognition by Western blot or immunoprecipitation analysis (by the methods described in Ausubel et al., supra). Antibodies may be used for TEM or immunofluorescent or other visualization techniques, for example, to visualize and study membrane fusion process. Antibodies which specifically recognize a sterically restricted antigen described herein are considered to be useful in the invention.

When the sterically restrictive agent (cargo) and linker both comprise amino acid sequences, a nucleic acid sequence encoding the sterically restricted N-trimer may be cloned into an expression cassette, which is driven by a promoter appropriate for the host cell and contains other transcriptional and translational signals necessary for expression of the sterically restricted N-trimer in the host cell. The sterically restricted N-trimer may then be expressed in mammalian cells using standard techniques known in the art. For example, the sterically restricted N-trimer may be placed under the control of a promoter, such as the Drosophila inducible metallothionein promoter and introduced into Drosophila cells. The sterically restricted N-trimer may also be followed by a poly (A) signal recognized by the host cell. Likewise, an Ab capable of recognizing a sterically restricted N-trimer may be produced using standard tools in molecular biology, which are well known in the art.

The steric block, steric agent, sterically restrictive agent, or cargo, may utilize glycosylation (“glycan shield”), polyethylene glycol (PEG), a protein (preferably, having a reduced immunogenicity or being non-immunogenic, e.g., albumin or other protein, which may be identified using IMMUNOPDA™ technology (from Xencor), or the method of Stickler et al. An in Vitro Human Cell-Based Assay to Rank the Relative Immunogenicity of Proteins, Toxicological Sciences (2003)), alkanes, alkanols, longer chain hydrocarbons, styrene-maleic acid copolymer, dextran, pyran copolymer, polylysine, polysaccharides, and inorganic compounds such as magnetite, gold and/or the like (see, for example, Proc. Natl. Acad. Sci. USA, vol. 84, pp. 1487-1491, 1981, and Biochemistry, vol. 28, pp. 6619-6624, 1989). Additional inaccessible antigens and antigens with reduced and restricted accessibility (38, 44, 45), are known in the art and may be used in the present invention. As will be recognized by a person of ordinary skill in the art using the guidance of the specification, the present invention provides for the use of mimics of an antigen, such as the the N-trimer region (e.g. 5-helix, IZN36, NCCG-gp41), that are modified by attachment of sterically restrictive agents, for example, proteins, addition of a flavonoid, polyethylene glycol, carbohydrate, a hydrocarbon chain from about 1 to about 400 carbons long, and/or inert particles, such that Abs and/or other compounds capable of penetrating a sterically restricted target are selected. The hydrocarbon chain may comprise a straight chain or branched, substituted or unsubstituted, alkyl, aryl, alkenyl, alkynyl, cycloalkyl, alkaryl, aralkyl group, or any combination thereof. Optionally, the stericly restrictive agent may be joined by way of a linker, for example, an amino acid sequence or other hydrocarbon chain.

As will be recognized in light of the present invention; such an antigen may be used to generate, boost, or screen for potent neutralizing Abs able to overcome the steric restriction. For example, the antigen may be administered to a subject as a vaccination against HIV or to induce an immunological response capable of reducing or removing a viral load in a previously infected subject.

Human immunodeficiency virus (HIV) entry is mediated by the viral envelope (Env) glycoprotein. As discussed, the gp41 ectodomain contains two helical heptad repeat sequences (N— and C-peptide regions) (1, 2). Peptides corresponding to these helical regions (N— and C-peptides) are dominant-negative inhibitors of HIV membrane fusion (2, 3). Isolated N— and C-peptides form a six-helix bundle (trimer-of-hairpins) when mixed in solution (4-6). In this structure, three N-peptides form a central parallel trimeric coiled coil (N-trimer) surrounded by three anti-parallel C-peptides that nestle between neighboring N-peptides.

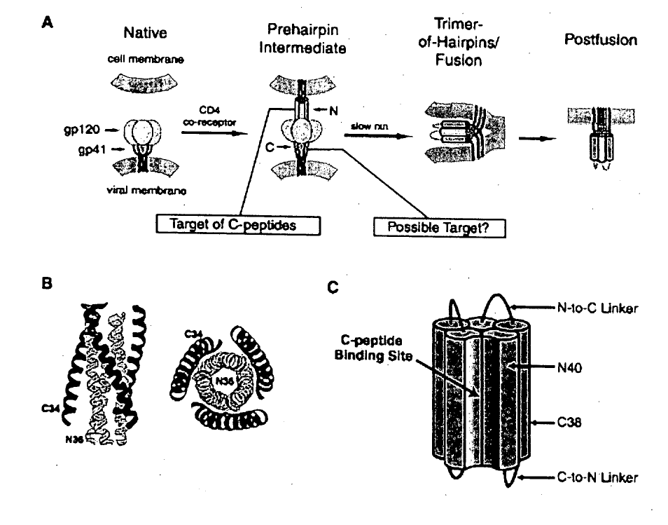

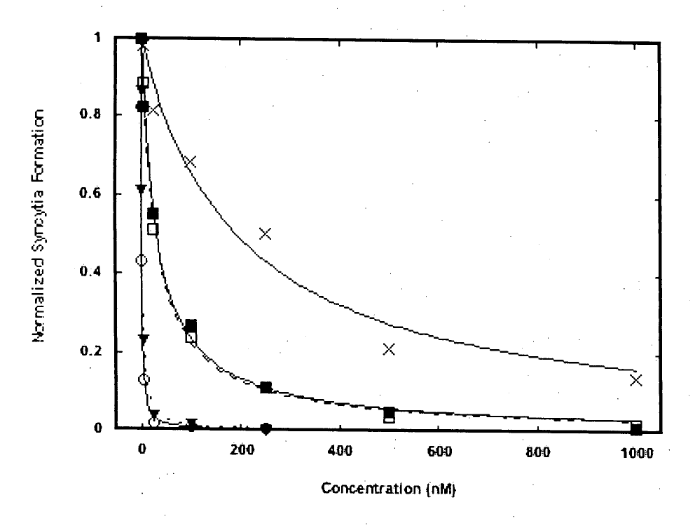

Based largely on these inhibitory and structural data, a working model of HIV-1 membrane fusion is proposed (FIG. 1) (3, 5). Initial interaction of Env with its target cell occurs via gp120 binding to CD4 and a coreceptor (typically CCR5 or CXCR4). This binding induces a series of large conformational changes in gp120 that are propagated to gp41 via the gp41-gp120 interface. At this stage, gp41 transiently adopts an extended “prehairpin intermediate” conformation that bridges both the viral and cellular membranes. This state is believed to persist for at least 15 min (3, 7, 8), but eventually collapses into a trimer-of-hairpins structure that pulls both membranes into tight apposition and induces membrane fusion (FIG. 1).

In this model, the prehairpin intermediate exposes the isolated N-trimer, whereas the C-peptide region exists in an unknown and possibly unstructured conformation remote from the N-trimer (3). At this stage, the prehairpin intermediate is vulnerable to binding of exogenous N— and C-peptides. Binding of the peptide inhibitors denies access of the endogenous N— or C-peptide regions to their appropriate intramolecular partners, thwarting hairpin formation and membrane fusion. This model predicts that any molecule that binds to the prehairpin intermediate and disrupts association of the N— and C-peptides will inhibit membrane fusion and has been successfully applied to the development of several potent entry inhibitors (9-11).

Designing peptide inhibitors of Env is a promising target for inhibition of viral infection. Env is the sole viral protein required for membrane fusion with the host cell by HIV-1. The N-trimer region of gp41 has been an important candidate for vaccine studies, but only two potent and broadly neutralizing antibodies against this region have yet been reported, 2F5 and 4E10, which bind just outside the C-terminal border of the C-peptide region, an area with uncertain structure. Hence, the C-terminal region of the gp41 ectodomain is an accessible target for the development of neutralizing antibodies, since this region appears to be partially exposed and vulnerable to an antiviral agent before, but the absence of structural constrain will hamper the ability to develop such neutralizing antibodies.

Additionally, the gp41 prehairpin intermediate has several promising features as an inhibitory target (12). Peptide mimics of the N-trimer region have been structurally characterized at high resolution (4-6). The interface between the N— and C-peptides is highly conserved among diverse HIV strains of both laboratory-adapted and clinical isolates (9). The N-trimer also presents a long (>100 Å) deep groove with an extensive binding surface (4-6). These special properties have led many groups to search for Abs that can disrupt this interface (reviewed in Ref. 13).

In principle, the gp41 N-trimer is an especially promising inhibition target, but despite the generation of numerous Abs with tight and specific binding against various mimics of the N-trimer, none of these Abs displays broadly neutralizing activity. This is because HIV has developed a strong steric defense against immune attack for this critical N-trimer region (Hamburger et al. (2005)). Thus, the gp41 N-trimer region has poor accessibility to large proteins. This defense may be a major factor in frustrating efforts to induce neutralizing Abs against the N-trimer region and may also explain why such neutralizing Abs against the N-trimer have not yet been observed in infected patients.

Biophysical experiments and structural studies have demonstrated that a C37 peptide (collectively referred to as “C-peptides”) inhibits viral fusion by binding along the full length of the surface groove of the N-trimer, including the deep hydrophobic “pocket” region previously shown to be an essential player in viral fusion. Inhibitors that specifically target this pocket have been developed (Eckert, D. M., and Kim, P. S. (2001) Proc. Natl. Acad. Sci. U.S.A. 98, 11187-11192). In addition, the fusion inhibitor T-20, a peptide based on the sequence of the C-peptide helix in gp41, blocks formation of the six-helix bundle and thus prevents membrane fusion (Chen, et al. 1995. A molecular clasp in the human immunodeficiency virus (HIV) type 1 TM protein determines the anti-HIV activity of gp41 derivatives: implication for viral fusion. J. Virol. 69:3771-3777; Matthews, et al. 2004. Enfuvirtide: the first therapy to inhibit the entry of HIV-1 into host CD4 lymphocytes. Nat. Rev. Drug Discov. 3:215-225, Wild, et al. 1994. Peptides corresponding to a predictive alpha-helical domain of human immunodeficiency virus type 1 gp41 are potent inhibitors of virus infection. Proc. Natl. Acad. Sci. USA 91:9770-9774).

C-peptides, which bind to the pre-hairpin intermediate, have shown reasonable success in human clinical trials as injected therapeutics (Kilby et al. 1998. Nat. Med. 4:1302-7). Participants who received 100 mg of the C-peptide T20 twice daily experienced viral reduction levels similar to patients treated with a reverse transcriptase or protease inhibitor. Therefore, inhibitors that block the formation of the timer-of-hairpins structure are currently under development as useful therapeutics.

However, there are disadvantages to the therapeutic use of C-peptides. First, because of their size, C-peptides are not amenable to oral routes of entry and must be injected. Second, large amounts of the peptide are required to observe an antiviral effect in humans. Therefore, the ability to raise an effective immunogenic response against the N-trimer in a subject represents a significant advancement in the field.

Although the HIV-1 Env protein is extremely immunogenic, attempts to raise potent neutralizing Abs in the laboratory against a broad range of HIV-1 viruses with viral and protein immunogens have been largely unsuccessful (Burton D R. 1997. Proc. Natl. Acad. Sci. USA 94:10018-23; Klein M. 1999. Vaccine 17:S65-70; Montefiori D C, and Evans T G. 1999. AIDS Res. Hum. Retroviruses 15:689-98). This is likely due to a variety of reasons. First, much of the sequence of Env is highly variable between viral strains, and therefore neutralizing antibodies are often strain-specific. Second, the high mutation rate of the virus likely allows quick escape from potentially neutralizing Abs. Finally, current antigens that mimic the N-timer are sterically open. Immunization with these antigens generates many antibodies that bind the antigen, but are unable to access their target region in HIV-1. Therefore, most protein immunogens used thus far have probably not properly represented the proper steric constraints necessary to elicit antibodies that will effectively neutralize HIV in vivo.

Only two reported anti-gp41 Abs potently neutralize a wide range of HIV-1 isolates, 2F5 and 4E10 (Burton et al. 1994. Science 266:1024-27; Li et al. 1997. AIDS Res. Hum. Retroviruses 13:647-56; Purtscher et al. 1994. AIDS Res. Hum. Retroviruses 10:1651-58; Roben et al. 1994. J. Virol. 68:4821-28; Trkola et al. 1995. J. Virol. 69:6609-17). Passive transfer of such antibodies can successfully protect Rhesus macaques against challenge by a SHIV containing either a laboratory-adapted or a primary isolate HIV-1 Env (Mascola J et al. 2000. Nat. Med. 6:207-10; Baba et al. 2000. Nat. Med. 6:200-6). However, neutralizing antibodies were administered at extremely high levels (ranging from 30-400 mg/kg) to observe this effect.

The present results suggest that HIV may have developed a strong steric defense against immune attack for its critical N-trimer region. Generally, we have shown that the gp41 N-trimer region has poor accessibility to large proteins. It is a logical extrapolation of the data presented here that a protein as large as IgG (150 kDa), even though it forms a somewhat elongated shape, will suffer a steric block at least as severe as observed with the largest protein, MBP (41 kDa), which is smaller than the individual (˜50 kDa) domains of an IgG. This defense may be a major factor in frustrating efforts to induce neutralizing Abs against the N-trimer region and may also explain why such neutralizing Abs against the N-trimer have not yet been observed in infected patients.

The steric restriction of the N-trimer stands in stark contrast to apparent accessibility of the extreme C-terminal region of the gp41 ectodomain (between the C-peptide region and the transmembrane domain). The only known potent and broadly neutralizing Abs against gp41 (2F5 and 4E10) target this region (22). Recent studies have suggested that this region may adopt a helical or β-strand conformation or cycle between the two (33, 39). For the most thoroughly studied Ab against this region, 2F5, a full-length IgG (˜150 kDa), is more potent than the Fab (˜50 kDa) (33), suggesting a freely accessible site. The dearth of effective antibodies against HIV-1, may be caused by the fact that the virus protects its conserved entry machinery with a steric block, preventing the binding of most antibodies. This is evidenced by the fact that all of the known fusion inhibitors that target the N-trimer in the pre-hairpin intermediate (e.g. C34, T-20, T-1249, D-peptides) are small (<40 residue) peptides and could circumvent the steric restriction of this structure.

There is also evidence suggesting that the C-peptide region may be more accessible than the N-trimer. The designed proteins 5-helix (25 kDa) (9) and N_{CCG}-gp41 (35 kDa) (40) target the C-peptide region and are potent entry inhibitors. Recently, a Pseudomonas endotoxin (PE) fusion with 5-helix (5-helix-PE, 65 kDa) was shown to inhibit viral entry with similar potency as 5-helix (41), although a toxic effect from PE may mask a loss of potency. Although the C-peptide region is likely accessible, it is difficult to target for vaccine studies, as it is unclear what organized structure (if any) this region adopts during viral entry.

C37 inhibits viral fusion by binding along the full length of the surface groove of the N-trimer, including the deep hydrophobic “pocket” region previously shown to be an essential player in viral fusion. Inhibitors that specifically target this pocket have been developed (10). The present invention contemplates the use of such pocket-specific inhibitors to circumvent the observed steric restriction. Cargo fused to the C terminus of C37 is believed to show a similar pattern of steric blockage and may also be used in the invention to generate Abs capable of avoiding the steric blockage of the N-trimer in vivo.

The steric restriction observe in the gp41 N-trimer is reminiscent of steric restrictions observed in gp120. These restrictions have been attributed to glycosylation (“glycan shield”) (42, 43) and/or inaccessible antigens (38, 44, 45). Previous studies with several broadly neutralizing gp120 Abs have shown that smaller versions of these Abs (Fabs or scFvs) often have significantly improved potency despite a loss of avidity (38, 46). The N-trimer steric restriction observed here may be more strict than seen in gp120, since proteins the size of Fabs (˜50 kDa) and scFvs (˜25 kDa) are already too large to fully access the gp41 N-trimer. Interestingly, the N-trimer region does not contain any glycosylation sites, probably because of its ultimate complete burial in the six-helix bundle structure. The N-trimer, however, may be affected by nearby glycosylation sites in gp120 or other regions of gp41 (the C-peptide region and N/C-peptide connecting loop are extensively glycosylated). A glycosylation site near the gp120 V3 loop has been shown to affect accessibility of the 2F5 Ab to its gp41 epitope in resistant strains (43).

These results suggest that attempts to improve the longevity of C-peptide inhibitors in the bloodstream may also be frustrated by steric issues. For instance, T-20, a 36-residue peptide recently approved by the FDA, is rapidly cleared from the bloodstream by kidney filtration, dramatically increasing dosing requirements. A reasonable approach for prevention of this rapid clearance is to cross-link C-peptide inhibitors to larger proteins (e.g. albumin) or high molecular weight polyethylene glycol, which also can reduce peptide immunogenicity (47). The present results suggest that these straightforward approaches will likely reduce the potency of modified C-peptides, and that use of smaller proteins or low molecular weight polyethylene glycol provide a more reasonable approach to the problem. The present invention further provides for longer linkers between a bulking group and the C-peptide inhibitor to improve accessibility to the N-trimer. Along with longer linkers, the present invention provides for stiffer (e.g. helical) linkers to provide better separation from large fusion partners and restore inhibitory potency. Hence, the present invention provides for longer and/or stiffer linkers between a C-peptide inhibitor and a bulking agent utilized to slow clearance of the inhibitor.

It should be noted that T-20, compared with C34, is derived from a gp41 sequence shifted about 10 amino acids toward the C-terminus and its binding site extends beyond the N-trimer region, which should be taken into consideration when designing a steric restriction or utilizing the protein according to the invention. Likewise, with the similar T-1249 inhibitor.

The present invention provides compounds and methods that may be used to discover and/or improve the chances of discovering a broadly neutralizing Ab against this valuable HIV target. Specifically, a designed, sterically restricted N-trimer antigen may be used to generate, boost, or screen for potent neutralizing Abs able to overcome the steric restriction. In an exemplary embodiment, the present invention provides mimics of the N-trimer region (e.g. N-peptide, 5-helix, IZN36, N_{CCG}-gp41, see also, 2, 3, 12, 14-16) modified by attachment to bulky proteins or large inert particles to select for Abs capable of penetrating a sterically recessed target.

Neutralizing Abs against sterically blocked gp120 targets often utilize unusually long CDR H3 loops to access recessed antigens (33, 46, 50). The insertion of longer linkers connecting MBP to C37 results in partial recovery of inhibitory activity, suggesting that extended CDR H3 loops may help penetrate the steric restriction on the gp41 N-trimer. These Abs are difficult to generate in small animals, as Abs in primates have longer CDR H3 loops on average than rodents (51). Therefore, one embodiment of the present invention provides for the generation of Abs having longer CDR loops. Potent N-trimer Abs may be more easily found using strategies that enrich for this type of Ab (e.g., engineered Ab libraries, Ab phage display, immunization of primates). Alternatively, very high affinity (sub-nM) Abs against the N-trimer may still be sufficiently neutralizing despite a substantial decrease in potency caused by the steric restriction.

Our results further suggest that the traditional depiction of the prehairpin intermediate as a symmetric structure (e.g. FIG. 1) may be inaccurate. The steric restriction of the N-trimer, and apparent accessibility of the C-peptide region, show that they reside in very different environments. Possible sources of this asymmetry include interactions with gp120, other regions of gp41, or cell surface proteins as well as glycosylation and differences between the curvature of the viral and cellular membranes.

The invention is further described by way of the following illustrative examples.

### EXAMPLES

### Example 1

### Protein Expression, Purification, and Characterization of IZN36 and Sterically Restricted C37

To test for steric constraints in accessing the gp41 N-trimer region a series of inhibitors containing a C-peptide attached to cargo proteins of various sizes were created. The cargo partners used were selected for the following properties: monomeric, soluble, globular, stable, tolerant to C-terminal additions, and free of non-specific peptide binding. Cargo proteins meeting these inclusion criteria and used to illustrate the invention range from 6 to 41 kDa (Table I). C37, the recombinant His-tagged version of the previously characterized synthetic peptide C34, was used as the reference inhibitor. In each fusion protein, C37 is connected at its N terminus to the C terminus of the cargo by a flexible 6- or 7-residue Ser/Gly linker. This linker was designed to be long enough to allow the proper orientation of C37 as it binds to the N-trimer but short enough for the attached cargo to prevent access to an occluded binding site. The N terminus of C37 was chosen for attachment of cargo because this attachment site points away from the membrane (whereas the C terminus of C37 is expected to be near the viral membrane and, therefore, less accessible). For each fusion protein, a matching control protein lacking C37 was also produced.

TABLE I
IC₅₀ (in nM) of fusion proteins in cell—cell fusion and viral infectivity assays

| | Fusion | | | | | | |
|---|---|---|---|---|---|---|---|
| | partner | | | | | | |
| | molecular | Cell- | IC₅₀ ratio | Viral | IC₅₀ | Viral | IC₅₀ |
| | mass | cell | (cell—cell | infectivity | ratio | infectivity | ratio |
| Protein | kDa | fusion | fusion) | HXB2 | HXB2 | JRFL | JRFL |
| C37 | 0 | 0.85 | 1.0 | 2.8 | 1.0 | 8.2 | 1.0 |
| BPTI-C37 | 6.5 | 1.5 | 1.8 | 3.1 | 1.1 | 4.8 | 0.6 |
| Ub-C37 | 8.6 | 4.7 | 5.5 | 6.8 | 2.5 | 37.7 | 4.6 |
| Mb-C37 | 17 | 30.8 | 36.2 | 58.0 | 21.0 | 414 | 50.5 |
| GFP-C37 | 27 | 28.9 | 34.0 | 118 | 42.8 | 533 | 65.0 |
| MBP-C37 | 41 | 192 | 225 | 206 | 74.8 | 1874 | 228 |
| MBP1-C37 | 41 | 75.1 | 88.4 | 88.9 | 32.2 | 640 | 78.0 |
| MBP2-C37 | 41 | 31.2 | 36.7 | 79.2 | 28.7 | 516 | 62.9 |

IC₅₀ S.E. is <25% for both assays. IC₅₀ ratios are relative to C37.

C37-H6 (C37), derived from the HXB2 Env sequence was expressed and purified. The proteins used were bovine pancreatic trypsin inhibitor (BPTI), human ubiquitin (Ub), sperm whale myoglobin (Mb), enhanced green fluorescent protein (GFP; Clontech), and Escherichia coli maltose-binding protein (MBP; New England Biolabs). Linker sequences were Ser4Gly2 for BPTI-C37, Ub-C37, and GFP-C37 and Ser5Gly2 for Mb-C37 and MBP-C37. The extended linker constructs had the following linker sequences: SSS(GGGS)3-SSSGG (MBP1-C37) (SEQ ID NO:5) and. SSS(GGGS)3S(GGGS)3SSSGG (MBP2-C37) (SEQ ID NO:6). The DNA encoding each protein was cloned into the following plasmids: pET9a (for BPTI-C37, Ub-C37, Mb-C37, and GFP-C37), pET20b (for BPTI-H6, Ub-H6, Mb-H6, and GFP-H6); pMAL-c2G (for MBP-H6, MBP-C37, MBP1-C37, and MBP2-C37).

Proteins were expressed in BL21(DE3)pLysS for pET9a and pET20b vectors and XL1-Blue for pMa1-c2G vectors. All proteins have C-terminal His tags (His6) and were purified using Ni affinity chromatography.

BPTI required refolding after expression for correct formation of disulfide bonds. Briefly, after Ni affinity purification, BPTI-C37-H6 and BPTI-H6 were reduced with 100 mM β-mercaptoethanol at pH 8 and dialyzed into 5% acetic acid. The proteins were air oxidized in the presence of a 1:10 ratio of oxidized:reduced glutathione at pH 8, 4° C. for 24 h. The correctly folded proteins were isolated using reverse phase HPLC and were confirmed by near-UV circular dichroism (Aviv 62DS) and measurement of trypsin inhibiting activity as previously described.

Cys-Gly-Gly-Asp-IZN36 is cloned into pET14b and expressed in BL21(DE3)pLysS. IZN36 was purified from inclusion bodies (solubilized in 6 M GuHCl) using Ni affinity chromatography. The protein was then dialyzed into 5% acetic acid and purified by reverse phase HPLC. This material was reduced with TCEP and biotinylated at its unique Cys residue using Biotin-HPDP. After biotinylation, the His tag was removed by thrombin cleavage, and the cleaved product was purified by reverse phase HPLC. The sequence of the final product is:

| | |
|---|---|
| GSHMCGGDIKKEIEAIKKEQEAIKKKIEAIEKEISGI | (SEQ ID NO:7) |
| VQQQNNLLRAIEAQQHLLQLTVWGIKQLQARIL. | |

### Example 2

### Surface Plasmon Resonance (SPR) Analysis of Sterically Restricted C37

Binding experiments were performed using a Biacore 2000 optical biosensor (University of Utah Protein Interaction Core Facility) equipped with research-grade CM5 sensor chips (Biacore). A standard coupling protocol was employed to immobilize streptavidin (SA; Pierce). Biotinylated IZN36 was captured on a SA surface, and free SA surfaces served as references.

Binding analysis of C37 and C37 fusion proteins was performed at 25° C. with a data collection rate of 2.5 Hz. The binding buffer (phosphate-buffered saline) +0.005% P20 detergent (Biacore)+1 mg/ml bovine serum albumin (fraction V; Fisher)) was prepared, vacuum filtered, and degassed immediately prior to use. Stock solutions of C37, C37 fusion proteins, and corresponding control proteins (without C37) were prepared in binding buffer at 100 nM. Protein binding was analyzed by injecting samples for 1 min over the IZN36 and reference surfaces using KINJECT at a flow rate of 50-100 μl/min. The dissociations were monitored for 3 min. The IZN36 surfaces were completely regenerated using one 3-s pulse of 6 M guanidine-HCl or three 6-s pulses of 0.1% SDS.

Data from the reference flow cells were subtracted to remove systematic artifacts that occurred in all flow cells. The data were normalized to the highest point in the response curve to facilitate comparison. Binding at one concentration was analyzed using a 1:1 binding model in CLAMP, assuming enough information from the curvature of the responses to determine the approximate kinetic parameters for the reactions.

The C-peptide Remains Accessible when Linked to Fusion Partners

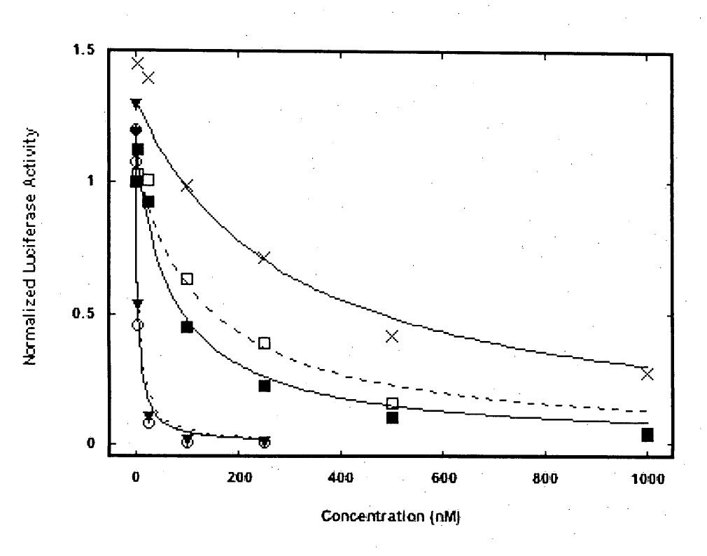

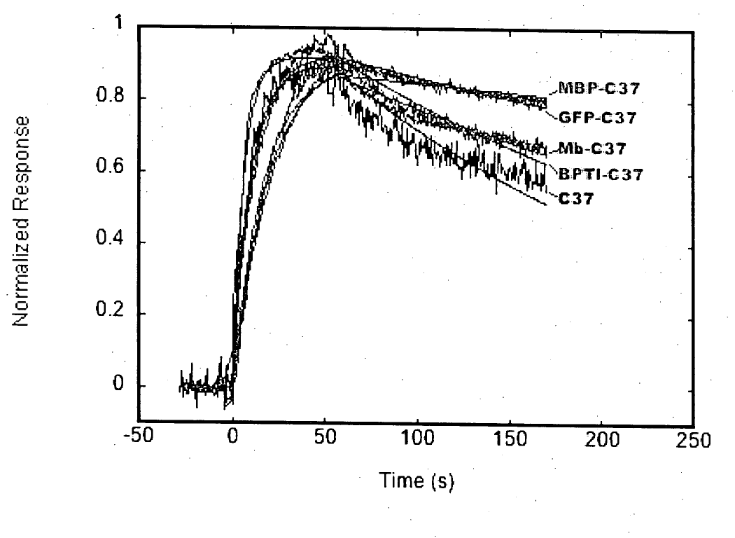

To ensure that linkage of C37-H6 to each of the partner proteins did not affect the accessibility of C37 for binding to a sterically open target, the fusion proteins and C37 were assayed for binding to IZN36, a soluble mimic of the N-trimer, using SPR. Each fusion protein was flowed over the control and IZN36 surfaces. C37 reversibly bound to IZN36 with a low nM K_{D} (FIG. 3). The calculated K_{D} for the fusion proteins are clustered in a narrow range around the C37 value (2-fold lower to 2-fold higher). The estimated kinetic parameters are similarly clustered, ranging from 3.2-fold slower to 1.4-fold faster (association rate) and up to 3.2-fold slower (dissociation rate). These rates are only approximate due to small systematic deviations from the fitting model, but as expected there is a slight trend toward slower association and dissociation rates with increasing molecular weight. These small differences in binding kinetics are likely responsible for some of the variation in potency observed here but rule out distinct binding kinetics as the major contributor to the substantial differences in potency among these inhibitors. These results also show that the accessibility and affinity of C37 are not significantly altered in the context of the fusion proteins. None of the cargo proteins alone showed measurable association with IZN36 at 100 nM (FIG. 2, inset).

In one embodiment of the invention, a cargo partner is fused to a C-peptide or a derivative thereof, or, preferably, an N-trimer mimic (e.g. 5-helix; IZN36; NCCG-gp41; those disclosed in U.S. Pat. Nos. 6,861,253; 6,821,723; 6,841,657; 6,818,740; 6,747,126; 6,737,067; 6,271,198; and variations or derivatives thereof), thereby producing a designed, sterically restricted, antigen that may be used to generate monoclonal antibodies or an immunological response in a subject, including, rabbits (e.g., for production of polyclonal antibodies), and a human (e.g., as a vaccine).

### Example 3

### Effects on Cell-Cell Fusion and Viral Infectivity Assay for Sterically Restricted C37

Cell-cell fusion was monitored. HXB2 Env-expressing Chinese hamster ovary cells were mixed with HeLa-CD4-LTR-β-galactosidase cells in the presence of inhibitors for 20 h at 37° C. Syncytia were stained with 5-bromo-4-chloro-3-indolyl-β-D-galactopyranoside (X-gal) and counted.

Viral infectivity was measured by the following method: pseudotyped viruses were produced by co-transfecting 293T cells using FuGENE (Roche Applied Science) with pNL4-3.Luc.R-E- and either pEBB-HXB2 or pEBB-JRFL. After 36-48 h, viral supernatants were collected and sterile filtered. HXB2 or JRFL pseudotyped virus was added to HOS-CD4-fusin or HOS-CD4-CCR5 cells, respectively, in the presence of inhibitors. HXB2 assays included 20 μg/ml DEAE-dextran. After 12 h, virus and inhibitor were removed and replaced with fresh media. Cells were lysed 40-44 h after infection using Glo lysis buffer (Promega), and luciferase activity was measured using Bright-Glo (Promega). IC50 values for both assays were calculated by fitting data to the equation, y=k/(1+[inhibitor]/IC₅₀), where y is the normalized number of syncytia or luciferase activity and k is the scaling constant.

Size and Inhibitory Potency Are Inversely Correlated

The inhibitory potency of each inhibitor was tested using the cell-cell fusion (syncytia) assay utilizing HXB2 Env and two viral infectivity assays utilizing either HXB2 (X4) or JRFL (R5) Envs (Table I, FIG. 2). C37 shows high potency inhibition in all assays (IC₅₀=0.85-8.2 nM). Inhibition is slightly weaker than seen with C34, as expected from the loss of helix-stabilizing synthetic blocking groups found in C34. The anti-gp41 Abs 2F5 and 4E10 have reported IC₅₀ values of ˜0.2-7 nM against HXB2/IIIB laboratory strains in cell-cell and viral infectivity assays similar to those used in this study.

The smallest fusion protein, BPTI-C37, also displays high potency in both assays, very similar to C37, demonstrating that the C37-cargo linker does not interfere with inhibitory activity. Ub-C37 is a slightly weaker (2.5-5.5-fold) inhibitor than C37, whereas Mb-C37 and GFP-C37 both show more substantial (21-65-fold) reductions in potency in both assays. MBP-C37 shows the most dramatic change with a 75-228-fold drop in potency. None of the control proteins (cargo without C37 peptide) inhibits at up to 1 μM (10 μM for MBP with JRFL Env) in either assay (data not shown).

In general, the cell-cell fusion and viral infectivity assays show similar losses of activity with increasing size of the inhibitors, with a slightly more pronounced effect on cell-cell fusion and JRFL-mediated viral entry. For HXB2 Env we observed up to a 4-fold greater potency in cell-cell fusion versus viral infectivity as seen in studies of other fusion inhibitors. As expected, inhibitors were less potent against the primary isolate JRFL in the viral infectivity assay. For most of the inhibitors, the viral infectivity data show a reproducible increase in infectivity (above the uninhibited values) at low inhibitor concentrations.

Partial Restoration of Inhibitory Potency with Extended Gly/Ser Linkers

To test whether a longer linker could overcome the steric restriction and restore inhibitory potency of the weakest inhibitor, we extended the flexible linker in MBP-C37 from its original length of 7 amino acids to 20 (MBP1-C37) or 33 (MBP2-C37) using Gly/Ser residues (Table I). Both extended linker inhibitors exhibit partial recovery of inhibitory potency. Compared with MBP-C37, MBP1-C37 and MBP2-C37 are 2.3-2.9-fold and 2.6-6.1-fold more potent, respectively (Table I). Compared with MBP-C37, MBPI- and MBP2-C37 interact similarly with IZN36 as measured by SPR (K_{D} vary by <20%, kₐ and k_{d} are <2-fold higher). In contrast to the other cargo-C37 fusions, a significant portion of the increased potency in MBP1- and MBP2-C37 may be attributable to an increased association rate.

### Example 4

### Stability of C37 Fusion Proteins During Fusion Assays

Inhibitors were analyzed for precipitation or extensive proteolysis to demonstrate that these processes did not cause the observed decrease in potency of the fusion proteins. C37 and the C37 fusions were incubated in tissue culture medium at 37° C. for 20 h to simulate the harshest conditions faced by the inhibitors during the cell-cell fusion and viral infectivity assays. Only trace (<2%) degradation was observed for all of the inhibitors (data not shown), allowing the conclusion that proteolysis did not cause a significant decrease in the potency of the inhibitors. However, the contribution of minor proteolytic breakdown products to increased inhibitory potency, particularly for the least potent inhibitors (1% contamination with free C37 would result in an apparent cell-cell fusion IC₅₀ value of ˜100 nM for a completely inactive inhibitor) should be considered by a person of ordinary skill in the art. An anti-His tag Western blot comparing samples before and after high speed centrifugation revealed no precipitation.

Likewise, proteolytic breakdown of a sterically restricted N-trimer mimic may result in the production of Abs directed to the sterically open N-trimer. Therefore, one embodiment of the invention provides for a cargo linked to an N-trimer mimic through a protease resistant linker, for example, a linker utilizing D amino acids, amide substitutions and other modifications known in the art to reduce proteolytic cleavage.

The C-peptide fusions used herein, demonstrate that the N-trimer region of gp41 is likely to be poorly accessible to proteins as large as Abs and provide a method of producing an antigen and/or effective Abs.

### Example 5

### Generation of IZN36-MBP and IZN36pm-MBP.

A chimeric recombinant DNA molecule encoding IZN36-MBP is created via the addition of MBP coding sequence and a Gly linker C-terminal of IZN36 which is already cloned into pET14b. Specifically, MBP coding sequence is operatively attached to the Gly linker which is operatively attached C-terminal end of the coding sequence of IZN36.

The pET14b containing the chimeric IZN36-MBP is inserted into BL21(DE3)pLysS and expressed to produce to chimeric protein. The IZN36-MBP is purified from bacterial lysate using Ni affinity chromatography. This material is reduced with TCEP and biotinylated at its unique Cys residue using Biotin-HPDP. After biotinylation, the His tag is removed by thrombin cleavage, and the cleaved product is purified by Ni affinity chromatography (unbound material is collected, while uncleaved material and cleaved His tags bind to the column).

IZN36pm-MBP is created and purified in essentially the same manner. However, IZN36pm-MBP has been engineered to contain a mutation in the pocket region of gp41 that prevent binding of antibodies that would normally bind to the pocket region.

### Example 6

### Assay Binding of C37 Based C-Peptide Inhibitors to IZN36-MBP

Binding experiments were performed using biotinylated IZN36-MBP is mixed with C37 or MBP-C37. This mixture is then added to magnetic streptavidin beads (Dynal), which bind to the biotinylated IZN36-MBP. These beads are precipitated magnetically, washed with TBS-T (TBS with 0.1% Tween-20), and analyzed by SDS-PAGE for the amount of C37 or MBP-C37 that co-precipitates with the biotinylated IZN36-MBP.

Results of these experiments indicate that IZN36-MBP allows the binding of C37, but has reduced binding for MBP-C37. This data indicates that the steric block of INZ36-MBP is reasonably similar to that of HIV on the C-terminal side.

In the alternative, binding experiments are performed using a Biacore 2000 optical biosensor (University of Utah Protein Interaction Core Facility) equipped with research-grade CM5 sensor chips (Biacore). A standard coupling protocol is employed to immobilize streptavidin (SA; Pierce). Biotinylated IZN36-MBP is captured on a SA surface, and free SA surfaces serve as references.

Binding analysis of C37 and MBP-C37 fusion protein is performed at 25° C. with a data collection rate of 2.5 Hz. The binding buffer (phosphate-buffered saline)+0.005% P20 detergent (Biacore)+1 mg/ml bovine serum albumin (fraction V; Fisher)) is prepared, vacuum filtered, and degassed immediately prior to use. Stock solutions of C37, MBP-C37 fusion protein, and corresponding control proteins (without C37) are prepared in binding buffer at 100 nM. Protein binding is analyzed by injecting samples for 1 min over the IZN36 and reference surfaces using KINJECT at a flow rate of 50-100 μl/min. The dissociations are monitored for 3 min. The IZN36-MBP surfaces are completely regenerated using one 3-s pulse of 6 M guanidine-HCI or three 6-s pulses of 0.1% SDS.

Data from the reference flow cells is subtracted to remove systematic artifacts that occurred in all flow cells. The data are normalized to the highest point in the response curve to facilitate comparison. Binding at one concentration is analyzed using a 1:1 binding model in CLAMP, assuming enough information from the curvature of the responses to determine the approximate kinetic parameters for the reactions.

### Example 7

### Phage Screening Using IZN36-MBP

Screening of a phage display peptide library is performed essentially as described previously (Miller et al. (2000) A human monoclonal antibody neutralizes divers HIV-1 isoaltes by binding a critical gp41 epitope, PNAS. 102(41):14759-14764) Libraries expressing antibodies or antibody fragments, preferably antibody fragments having longer CDR3 regions are used, such as the phage antibody library available from Merck Research Labs. A pool of these libraries, for example, containing 10¹¹-10¹² infectious particles is screened, for example, with IZN36-MBP. Briefly, the selection strategy is designed to isolate cross-specific scFvs from large naive scFv libraries is based upon methods described (53). Phage supernatants are screened by bacteriophage ELISA as described (54, 55), where the biotinylated form of IZN36-MBP are immobilized onto a 96-well strepdavidin plates. As a source of antibodies, a large diverse well characterized library of bacteriophage bearing scFvs derived from normal human B cells may be used (53). From a starting population, target-specific scFvs are obtained after two rounds of sequential selection for binding to the biotinylated form of IZN36-MBP. The scFvs are then sequenced to determine the number of unique sequences. Using an HIVRP assay (57), purified scFvs from the IZN36-MBP binding bacteriophage are screened and an scFv that blocks viral entry is identified.

Using this method antibodies in the library which are capable of binding to IZN36pm-MBP are determined. As these antibodies do not bind to the pocket region that is of greatest interest, these antibodies can be removed from the screen as binding to areas on IZN36-MBP that are not of the highest interest. Further, the antibody library can be pre-screened for antibodies that bind to IZN36 alone. Once these preliminary steps are accomplished, IZN36-MBP can be screened against those antibodies that bind to IZN36 alone but are unable to bind to IZN36pm-MBP. Antibodies identified through this screen are then expressed and purified using standard techniques.

### Example 8

### Optimization of Antigenic Epitope in IZN36-MBP

Antibodies identified as binding to IZN36-MBP in Examples 7 and/or 10 will be crystallized in complex with IZN36-MBP. This structure will provide data on how to improve the design of the sterically blocked antigen to more selectively induce neutralizing antibody responses. Improved designs of the sterically blocked antigens may be used in conjunction with the other Examples defined herein to, for example, generate or identify binding antibodies and to prevent HIV infection.

### Example 9

### Optimization of steric agent linked to IZN36

The steric agent linked to IZN36 is optimized to behave as an N-trimer. Briefly, the ability of the various C37 based chimeras described in Example 1 will be tested for their ability to bind to an IZN36 linked to a steric agent using, for example, the protocols outlined in Example 6. The steric agent can then be adjusted and retested repeatedly until the combination of IZN36 and the steric agent behaves, with regards to the various C37 based chimeras, in a matter similar to that of the wild type N-trimer region.

### Example 10

### Animal Based Generation of Antibodies to IZN36-MBP

Purified IZN36-MBP is used to generate antibodies in rabbits and rhesus monkeys using methods well known in the art. Serum from the animals is obtained and antibodies to IZN36-MBP are purified using standard techniques

### Example 11

### Inhibition of HIV Infection Using Antibodies That Bind to IZN36-MBP

Cell-cell fusion is monitored. HXB2 Env-expressing Chinese hamster ovary cells are mixed with HeLa-CD4-LTR-β-galactosidase cells in the presence of the antibodies to IZN36-MBP identified in Examples 7 and/or 9. for 20 h at 37° C. Syncytia are stained with 5-bromo-4-chloro-3-indolyl-β-D-galactopyranoside (X-gal) and counted.

Viral infectivity is measured by the following method: pseudotyped viruses are produced by co-transfecting 293T cells using FuGENE (Roche Applied Science) with pNL4-3.Luc.R-E- and either pEBB-HXB2 or pEBB-JRFL. After 36-48 h, viral supernatants are collected and sterile filtered. HXB2 or JRFL pseudotyped virus is added to HOS-CD4-fusin or HOS-CD4-CCR5 cells, respectively, in the presence of antibodies to IZN36-MBP. HXB2 assays includ 20 μg/ml DEAE-dextran. After 12 h, virus and antibodies to IZN36-MBP identified in Examples 7 and/or 9 are removed and replaced with fresh media. Cells are lysed 40-44 h after infection using Glo lysis buffer (Promega), and luciferase activity is measured using Bright-Glo (Promega). IC50 values for both assays are calculated by fitting data to the equation, y=k/(1+[inhibitor]/IC₅₀), where y is the normalized number of syncytia or luciferase activity and k is the scaling constant.

Antibodies to IZN36-MBP identified in Examples 7 and/or 9 that are able to attenuate or inhibit HIV infection are thus identified.

### Example 12

### Treatment of HIV Infected Subjects with Antibodies to IZN36-MBP

A subject, such as a primate, that has an ongoing HIV infection is provided. The viral titer of the subject is determined. The subject is then treated under various dosage regimes with a pharmaceutical composition comprising one or more of the antibodies identified in Example 10. After 1, 2, 4, and 8 weeks of treatment, viral titers are again determined from the subjects. The treatment with the pharmaceutical composition is shown to attenuate the HIV infection.

### Example 13

### Prophylactic Treatment of Subjects Exposed to HIV with Antibodies to IZN36-MBP

A subject, such as a primate, that has been exposed to HIV or is believed to be at risk of developing an HIV infection is provided. The subject is then treated under various dosage regimes with a pharmaceutical composition comprising one or more of the antibodies identified in Example 10. After 1, 2, 4, and 8 weeks of treatment, viral titers are again determined from the subject. The treatment with the pharmaceutical composition is shown to prevent HIV infection.

### Example 14

### Treatment of HIV Infected Subjects with IZN36-MBP

A subject, such as a primate, that has an ongoing HIV infection is provided. The viral titer of the subject is determined. The subject is then treated under various dosage regimes with a pharmaceutical composition comprising IZN36-MBP. After 1, 2, 4, and 8 weeks of treatment, viral titers are again determined from the subjects. The treatment with the pharmaceutical composition is shown to attenuate the HIV infection.

### Example 15

### Prophylactic Treatment of Subjects Exposed to HIV with IZN36-MBP

A subject, such as a primate, that has been exposed to HIV or is believed to be at risk of developing an HIV infection is provided. The subject is then treated under various dosage regimes with a pharmaceutical composition comprising IZN36-MBP. After 1, 2, 4, and 8 weeks of treatment, viral titers are again determined from the subject. The treatment with the pharmaceutical composition is shown to prevent HIV infection.

### Example 16

### Immunization of Subjects to HIV Infection with IZN36-MBP

A subject, such as a primate, is provided. The subject is then treated under various dosage regimes with a pharmaceutical composition comprising IZN36-MBP. After 1, 2, 4, or 8 weeks serum is obtained from the subject and the presence of antibodies directed to IZN36-MBP determined. The treatment with the pharmaceutical composition is shown to immunize the subject against HIV infection.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

### REFERENCES

All references, including publications, patents, and patent applications, cited herein are hereby incorporated by reference to the same extent as if each reference were individually and specifically indicated to be incorporated by reference and were set forth in its entirety herein.
1. Lu, M., Blacklow, S. C., and Kim, P. S. (1995) Nat. Struct. Biol. 2, 1075-1082;
2. Wild, C. T., Shugars, D. C., Greenwell, T. K., McDanal, C. B., and Matthews, T. J. (1994) Proc. Natl. Acad. Sci. U.S.A. 91, 9770-9774;
3. Chan, D. C., and Kim, P. S. (1998) Cell 93, 681-684;
4. Tan, K., Liu, J., Wang, J., Shen, S., and Lu, M. (1997) Proc. Natl. Acad. Sci. U.S.A. 94, 12303-12308;
5. Weissenhom, W., Dessen, A., Harrison, S. C., Skehel, J. J., and Wiley, D. C. (1997) Nature 387, 426-430;
6. Chan, D. C., Fass, D., Berger, J. M., and Kim, P. S. (1997) Cell 89, 263-273;
7. Munoz-Barroso, I., Durell, S., Sakaguchi, K., Appella, E., and Blumenthal, R. (1998) J. Cell Biol. 140,315-323;
8. Melikyan, G. B., Markosyan, R. M., Hemmati, H., Delmedico, M. K., Lambert, D. M., and Cohen, F. S. (2000) J. Cell Biol. 151, 413-423;
9. Root, M. J., Kay, M. S., and Kim, P. S. (2001) Science 291, 884-888;
10. Eckert, D. M., and Kim, P. S. (2001) Proc. Natl. Acad. Sci. U.S.A. 98, 11187-11192;
11. Eckert, D. M., Malashkevich, V. N., Hong, L. H., Carr, P. A., and Kim, P. S. (1999) Cell 99, 103-115;
12. Eckert, D. M., and Kim, P.S. (2001) Annu. Rev. Biochem. 70,777-810;
13. Burton, D. R., Desrosiers, R. C., Doms, R. W., Koff, W. C., Kwong, P. D., Moore, J. P., Nabel, G. J., Sodroski, J., Wilson, I. A., and Wyatt, R. T. (2004) Nat. Immunol. 5, 233-236;
14. Wild, C., Oas, T., McDanal, C., Bolognesi, D., and Matthews, T. (1992) Proc. Natl. Acad. Sci. U.S.A. 89, 10537-10541;
15. Wild, C., Greenwell, T., and Matthews, T. (1993) AIDS Res. Hum. Retroviruses 9, 1051-1053;
16. Jiang, S., Lin, K., Strick, N., and Neurath, A. R. (1993) Nature 365, 113;
17. Weiss, C. D. (2003) AIDS Rev. 5, 214-221;
18. Rimsky, L. T., Shugars, D. C., and Matthews, T. J. (1998) J. Virol. 72, 986-993;
19. Golding, H., Zaitseva, M., de Rosny, E., King, L. R., Manischewitz, J., Sidorov, I., Gorny, M. K., Zolla-Pazner, S., Dimitrov, D. S., and Weiss, C. D. (2002) J. Virol. 76, 6780-6790;
20. Opalka, D., Pessi, A., Bianchi, E., Ciliberto, G., Schleif, W., McElhaugh, M., Danzeisen, R., Geleziunas, R., Miller, M., Eckert, D. M., Bramhill, D., Joyce, J., Cook, J., Magilton, W., Shiver, J., Emini, E., and Esser, M. T. (2004) J. Immunol. Methods 287, 49-65;
21. Louis, J. M., Nesheiwat, I., Chang, L., Clore, G. M., and Bewley, C. A. (2003) J. Biol. Chem. 278, 20278-20285;
22. McGaughey, G. B., Barbato, G., Bianchi, E., Freidinger, R. M., Garsky, V. M., Hurni, W. M., Joyce, J. G., Liang, X., Miller, M. D., Pessi, A., Shiver, J. W., and Bogusky, M. J. (2004) Curr. HIV Res. 2, 193-204;
23. Chen, B. K., Saksela, K., Andino, R., and Baltimore, D. (1994) J. Virol. 68, 654-660;
24. Coplen, L. J., Frieden, R. W., and Goldenberg, D. P. (1990) Proteins 7, 16-31;
25. Edelhoch, H. (1967) Biochemistry 6, 1948-1954;
26. Johnsson, B., Lofas, S., and Lindquist, G. (1991) Anal. Biochem. 198, 268-277;
27. Myszka, D. G. (1999) J. Mol. Recognit. 12, 279-284;
28. Myszka, D. G., and Morton, T. A. (1998) Trends Biochem. Sci. 23, 149-150;
29. Canziani, G. A., Klakamp, S., and Myszka, D. G. (2004) Anal. Biochem. 325, 301-307;
30. Chan, D. C., Chutkowski, C. T., and Kim, P. S. (1998) Proc. Natl. Acad. Sci. U.S.A. 95, 15613-15617;
31. Kozarsky, K., Penman, M., Basiripour, L., Haseltine, W., Sodroski, J., and Krieger, M. (1989) J. Acquir. Immune Defic. Syndr. 2, 163-169;
32. Malashkevich, V. N., Chan, D. C., Chutkowski, C. T., and Kim, P. S. (1998) Proc. Natl. Acad. Sci. U.S.A. 95, 9134-9139;
33. Zwick, M. B., Komori, H. K., Stanfield, R. L., Church, S., Wang, M., Parren, P. W., Kunert, R., Katinger, H., Wilson, I. A., and Burton, D. R. (2004) J. Virol. 78, 3155-3161;
34. Stiegler, G., Kunert, R., Purtscher, M., Wolbank, S., Voglauer, R., Steindl, F., and Katinger, H. (2001) AIDS Res. Hum. Retroviruses 17, 1757-1765;
35. Sia, S. K., and Kim, P. S. (2003) Proc. Natl. Acad. Sci. U.S.A. 100, 9756-9761;
36. Wei, X., Decker, J. M., Liu, H., Zhang, Z., Arani, R. B., Kilby, J. M., Saag, M. S., Wu, X., Shaw, G. M., and Kappes, J. C. (2002) Antimicrob. Agents Chemother. 46, 1896-1905;
37. Reeves, J. D., Gallo, S. A., Ahmad, N., Miamidian, J. L., Harvey, P. E., Sharron, M., Pohlmann, S., Sfakianos, J. N., Derdeyn, C. A., Blumenthal, R., Hunter, E., and Doms, R. W. (2002) Proc. Natl. Acad. Sci. U.S.A. 99, 16249-16254;
38. Labrijn, A. F., Poignard, P., Raja, A., Zwick, M. B., Delgado, K., Franti, M., Binley, J., Vivona, V., Grundner, C., Huang, C. C., Venturi, M., Petropoulos, C. J., Wrin, T., Dimitrov, D. S., Robinson, J., Kwong, P. D., Wyatt, R. T., Sodroski, J., and Burton, D. R. (2003) J. Virol. 77, 10557-10565;
39. Barbato, G., Bianchi, E., Ingallinella, P., Hurni, W. H., Miller, M. D., Ciliberto, G., Cortese, R., Bazzo, R., Shiver, J. W., and Pessi, A. (2003) J. Mol. Biol. 330, 1101-1115;
40. Louis, J. M., Bewley, C. A., and Clore, G. M. (2001) J. Biol. Chem. 276, 29485-29489;
41. Root, M. J., and Hamer, D. H. (2003) Proc. Natl. Acad. Sci. U.S.A. 100, 5016-5021;
42. Wei, X., Decker, J. M., Wang, S., Hui, H., Kappes, J. C., Wu, X., Salazar-Gonzalez, J. F., Salazar, M. G., Kilby, J. M., Saag, M. S., Komarova, N. L., Nowak, M. A., Hahn, B. H., Kwong, P. D., and Shaw, G. M. (2003) Nature 422, 307-312;
43. McCaffrey, R. A., Saunders, C., Hensel, M., and Stamatatos, L. (2004) J. Virol. 78, 3279-3295;
44. Kwong, P. D., Wyatt, R., Robinson, J., Sweet, R. W., Sodroski, J., and Hendrickson, W. A. (1998) Nature 393, 648-659;
45. Saphire, E. O., Parren, P. W., Pantophlet, R., Zwick, M. B., Morris, G. M., Rudd, P. M., Dwek, R. A., Stanfield, R. L., Burton, D. R., and Wilson, I. A. (2001) Science 293, 1155-1159;
46. Choe, H., Li, W., Wright, P. L., Vasilieva, N., Venturi, M., Huang, C. C., Grundner, C., Dorfinan, T., Zwick, M. B., Wang, L., Rosenberg, E. S., Kwong, P. D., Burton, D. R., Robinson, J. E., Sodroski, J. G., and Farzan, M. (2003) Cell 114, 161-170;
47. Harris, J. M., and Chess, R. B. (2003) Nat. Rev. Drug. Discov. 2, 214-221;
48. Reeves, J. D., Miamidian, J. L., Biscone, M. J., Lee, F. H., Ahmad, N., Pierson, T. C., and Doms, R. W. (2004) J. Virol. 78, 5476-5485;
49. Kliger, Y., Gallo, S. A., Peisajovich, S. G., Munoz-Barroso, I., Avkin, S., Blumenthal, R., and Shai, Y. (2001) J. Biol. Chem. 276, 1391-1397;
50. Zwick, M. B., Parren, P. W., Saphire, E. O., Church, S., Wang, M., Scott, J. K., Dawson, P. E., Wilson, I. A., and Burton, D. R. (2003) J. Virol. 77, 5863-5876;
51. Wu, T. T., Johnson, G., and Kabat, E. A. (1993) Proteins 16, 1-7;
52. Miller, M., and Geleziunas, R. (2004) Abstracts of the 13th International HIV Drug Resistance Workshop, Tenerife Sur-Costa Adeje, Canary Islands, Spain, Jun. 8-12, 2004 Abstract 9, International Medical Press, Ltd., London;
53. Vaughan et al., (1996) Nat. Biotechnology, 14, 309-314;
54. McCafferty et al., (1990) Nature, 348, 552-554;
55. Lou et al., (2001) J. Immunol. Methods, 253, 233-242;
56. Osbourn et al., (1996) Immunotechnology, 2, 181-196; and
57. Tobiume et al., (2003) J. Virol., 77, 10645-10650.

What is claimed is:

1. An isolated sterically restricted antigen comprising an antigen linked to a steric agent that restricts free access to the antigen.

2. The sterically restricted antigen of claim 1, wherein the antigen is associated with a disease state.

3. The sterically restricted antigen of claim 1, wherein the antigen is a mimic of the N-trimer region of gp41.

4. The sterically restricted antigen of claim 3, wherein the antigen cannot be bound by an antibody that can bind an isolated N-trimer region of gp41, but not to a wild type N-trimer region of gp41.

5. The sterically restricted antigen of claim 1, wherein the antigen is selected from the group consisting of IZN17, IZN36, 5-helix, IZN36, N-peptide, N34_{CCG}-N13 and N_{CCG}-gp41.

6. The sterically restricted antigen of claim 1, wherein the steric agent has a size from about 20 Å to about 10 um, and wherein the steric agent blocks free access to the antigen.

7. The sterically restricted antigen of claim 1, wherein the steric agent is selected from the group consisting of Maltose Binding Protein, serum albumin, ubiquitin, Streptavidin, immunoglobulin domains, keyhole limpet hemacyanin, sperm whale myoglobin, bovine pancreatic trypsin inhibitor, gold particles, magnetic particles, Green Fluorescent Protein, glycans, and polyethylene glycol.

8. The sterically restricted antigen of claim 1 wherein the antigen is IZN36 and the steric agent is Maltose Binding Protein.

9. The sterically restricted antigen of claim 1, wherein the steric agent is a modified peptide.

10. The sterically restricted antigen of claim 9, wherein the modification comprises adding polyethylene glycol to the peptide.

11. The sterically restricted antigen of claim 1, wherein the steric agent comprises a hydrocarbon.

12. The sterically restricted antigen of claim 1, wherein the steric agent is a liposome and wherein the antigen is linked to the steric agent by embedding the antigen in the liposome.

13. The sterically restricted antigen of claim 1, wherein the steric agent comprises a polysaccharide.

14. The sterically restricted antigen of claim 1, wherein the steric agent is linked to the antigen via a linker.

15. The sterically restricted antigen of claim 14, wherein where the linker comprises from about 1 to about 40 units of Ser and/or Gly.

16. An isolated antibody directed against the sterically restricted antigen of claim 1.

17. The isolated antibody of claim 16, wherein the antibody comprises a CDR3 region sufficiently long enough to overcome a steric block.

18. The isolated antibody of claim 16, wherein the antibody binds to the antigen portion of the sterically restricted antigen.

19. The antibody of claim 18, wherein the binding of the antibody to a molecule from which the antigen is derived prevent the function of the molecule from which the antigen is derived.

20. The isolated antibody of claim 16, wherein the antibody is identified by a process comprising:
providing the sterically restricted antigen of claim 1 to a culture of cells producing an antibody or phage capable of expressing the binding region of an antibody; and
assaying for binding of the antibody to the sterically restricted antigen.

21. The process according to claim 20, further comprising identifying the antibody.

22. The method according to claim 20, comprising providing the sterically restricted antigen to a phage display library capable of expressing an antibody.

23. The method according to claim 22, wherein providing a sterically restricted antigen comprises providing a sterically restricted N-trimer mimic.

24. A method of treating or preventing a disease in a subject, the method comprising:
providing to the subject a pharamaceutical composition comprising the antibody of claim 16; and
monitoring the presence of the disease state in the subject.

25. The method according to claim 24, wherein the disease is AIDS.

26. A method of inducing an antigenic response to a sterically restricted antigen, the method comprising:
administering a pharmaceutical composition comprising the sterically restricted antigen of claim 1 to a subject; and
producing an antibody response to the sterically restricted antigen in the subject.

27. The method according to claim 26, wherein the antigen portion of the sterically restricted antigen is associated with a disease.

28. The method according to claim 27, wherein the produced antibody response treats the disease or prevents infection with the disease.

29. The method according to claim 27, wherein the produced antibody response immunizes the subject against the disease.

30. The method according to claim 27, wherein the disease is AIDS.

31. The method according to claim 26, further comprising isolating the antibody from the subject.

32. The method according to claim 26, wherein the subject is a primate.


SEQUENCE LISTING (7 sequences)

SEQ ID NO: 1 - protein, 40 aa, Artificial
  N-peptide segment of 5-helix
QLLSGIVQQQNNLLRAIEAQQHLLQLTVWGIKQLQARILA

SEQ ID NO: 2 - protein, 38 aa, Artificial
  C-peptide segment of 5-helix
HTTWMEWDREINNYTSLIHSLIEESQNQQEKNEQELLE

SEQ ID NO: 3 - protein, 5 aa, Artificial
  Linker
GGSGG

SEQ ID NO: 4 - protein, 5 aa, Artificial
  Linker
GSSGG

SEQ ID NO: 5 - protein, 20 aa, Artificial
  Linker
SSSGGGSGGGSGGGSSSSGG

SEQ ID NO: 6 - protein, 33 aa, Artificial
  Linker
SSSGGGSGGGSGGGSSGGGSGGGSGGGSSSSGG

SEQ ID NO: 7 - protein, 70 aa, Artificial
  IZN36
GSHMCGGDIKKEIEAIKKEQEAIKKKIEAIEKEISGIVQQQNNLLRAIEAQQHLLQLTVWGIKQLQARIL